(19) United States
(12) Patent Application Publication
(10) Pub. No.: US 2018/0256635 A1
(43) Pub. Date: Sep. 13, 2018

(54) LAXATIVE FORMULATIONS AND MANUFACTURE

(71) Applicant: COLONARYCONCEPTS LLC, Palm Beach Gardens, FL (US)

(72) Inventors: Michael ALLIO, Providence, RI (US); Jonathan GORDON, Narragansett, RI (US)

(21) Appl. No.: 15/756,922

(22) PCT Filed: Sep. 1, 2016
(86) PCT No.: PCT/US16/50005

§ 371 Date: Mar. 1, 2018

Related U.S. Application Data
(60) Provisional application No. 62/212,701, filed on Sep. 1, 2015.

(51) Int. Cl. A61K 31/765; A23L 33/115; A23L 2/52

(52) U.S. Cl. CPC A61K 31/765; A23V 2002/00; A23L 2/52; A23L 33/115

(57) ABSTRACT

Described herein are compositions for purging the GI tract of a subject together with methods of manufacture of said compositions. In various embodiments, compositions for purging the GI tract of a subject may comprise USP food ingredients and USP/NF grade chemicals mixed together to form homogeneous edible mixtures. Methods of manufacture of formulations for delivery laxatives may comprise a temperature controlled process.

### CROSS-REFERENCE

This application claims the benefit of U.S. Provisional Application No. 62/212,701 filed Sep. 1, 2015, which is incorporated by reference herein in its entirety.

### BACKGROUND

Laxatives may be taken by a subject for a number of reasons. Laxatives are, for example, prescribed for certain medical indications such as the cleansing of the GI tract in preparation for a medical or surgical procedure.

There are numerous types of laxatives, which have a number of different mechanisms of action. Polyethylene glycol, which is also known as PEG, is an osmotic laxative that is used for irrigation of the colon and GI tract in preparation for medical and surgical procedures.

PEG-3350 is sold in the U.S. in laxative compositions that typically are to be combined with a liquid to form a solution. Liquid formulations of PEG are inconvenient, unsatisfying, and uncomfortable to the consumer: they suffer from poor taste, an unpleasant, viscous and salty texture and mouthfeel, and/or create a bloated, overwhelmed feeling in the consumer's stomach. Undesirable mouth-feel is at least in part caused by the osmotic effects of PEG in the mouth and pharynx when ingested.

### SUMMARY

Described herein are compositions for purging the GI tract of a subject, compositions for treating or relieving constipation in a subject, as well as methods of manufacture of said compositions.

Described herein is a composition containing PEG 3350 and one or more USP/NF grade food ingredients, wherein the composition comprises an essentially homogeneous mixture of the PEG 3350 and the one or more USP/NF grade food ingredients. In some embodiments, the composition comprises at least 10 g of PEG 3350. In some embodiments, the composition comprises at least 12 g of PEG 3350. In some embodiments, the composition comprises at least 15 g of PEG 3350. In some embodiments, the composition comprises at least 20 g of PEG 3350. In some embodiments, the composition comprises at least 25 g of PEG 3350. In some embodiments, the composition comprises at least 30 g of PEG 3350. In some embodiments, the composition comprises at least 35 g of PEG 3350. In some embodiments, the composition comprises at least 40 g of PEG 3350. In some embodiments, the composition comprises at least 45 g of PEG 3350. In some embodiments, the composition comprises about 50% PEG 3350 by weight. In some embodiments, the composition comprises about 55% PEG 3350 by weight. In some embodiments, the composition comprises about 60% PEG 3350 by weight. In some embodiments, the composition comprises about 65% PEG 3350 by weight. In some embodiments, the composition comprises about 70% PEG 3350 by weight. In some embodiments, the composition comprises about 75% PEG 3350 by weight. In some embodiments, the composition comprises about 80% PEG 3350 by weight. In some embodiments, the total weight of the composition comprises about 65 g. In some embodiments, the total weight of the composition comprises about 43 g.

Described herein are methods for manufacturing a composition comprising the steps of heating one or more USP/NF grade food ingredients to a temperature of about 90 degrees Fahrenheit until the one or more USP/NF grade food ingredients is essentially liquefied; and mixing the essentially liquefied one or more USP/NF grade food ingredients with a laxative. In some embodiments, at least one of the one or more USP/NF grade food ingredients comprises a pure fat. In some embodiments, at least one of the one or more USP/NF grade food ingredients is a solid at room temperature. In some embodiments, at least one of the one or more USP/NF grade food ingredients comprises cocoa butter. In some embodiments, the composition comprises syrup. In some embodiments, herein the step of mixing comprises mixing the one or more USP/NF grade food ingredients with the laxative at a temperature below 70 degrees Fahrenheit. In some embodiments, the laxative comprises PEG 3350. In some embodiments, the laxative comprises at least 10 g of PEG 3350. In some embodiments, the laxative comprises at least 12 g of PEG 3350. In some embodiments, the laxative comprises at least 15 g of PEG 3350. In some embodiments, the laxative comprises at least 20 g of PEG 3350. In some embodiments, the laxative comprises at least 25 g of PEG 3350. In some embodiments, the laxative comprises at least 30 g of PEG 3350. In some embodiments, the laxative comprises at least 35 g of PEG 3350. In some embodiments, the laxative comprises at least 40 g of PEG 3350. In some embodiments, the laxative comprises at least 45 g of PEG 3350. In some embodiments, the composition comprises about 50% PEG 3350 by weight. In some embodiments, the laxative comprises about 55% PEG 3350 by weight. In some embodiments, the composition comprises about 60% PEG 3350 by weight. In some embodiments, the composition comprises about 65% PEG 3350 by weight. In some embodiments, the composition comprises about 70% PEG 3350 by weight. In some embodiments, the composition comprises about 75% PEG 3350 by weight. In some embodiments, the composition comprises about 80% PEG 3350 by weight. In some embodiments, the total weight of the composition comprises about 65 g. In some embodiments, the total weight of the composition comprises about 43 g.

Described herein is composition comprising an essentially homogeneous mixture of a laxative and one or more USP/NF food ingredients wherein the at least one of the one or more USP/NF food ingredients are in essentially solid form at room temperature and essentially liquid form at body temperature. In some embodiments, the laxative comprises PEG 3350. In some embodiments, the laxative comprises at least 10 g of PEG 3350. In some embodiments, the laxative comprises at least 12 g of PEG 3350. In some embodiments, the laxative comprises at least 15 g of PEG 3350. In some embodiments, the laxative comprises at least 20 g of PEG 3350. In some embodiments, the laxative comprises at least 25 g of PEG 3350. In some embodiments, the laxative comprises at least 30 g of PEG 3350. In some embodiments, the laxative comprises at least 35 g of PEG 3350. In some embodiments, the laxative comprises at least 40 g of PEG 3350. In some embodiments, the laxative comprises at least 45 g of PEG 3350. In some embodiments, the composition comprises about 50% PEG 3350 by weight. In some embodiments, the composition comprises about 55% PEG 3350 by weight. In some embodiments, the composition comprises about 60% PEG 3350 by weight. In some embodiments, the composition comprises about 65% PEG 3350 by weight. In some embodiments, the composition comprises about 70% PEG 3350 by weight. In some embodiments, the composition comprises about 75% PEG 3350 by weight. In some embodiments, the composition comprises about 80% PEG 3350 by weight. In some embodiments, the total weight of the composition comprises about 65 g. In some embodiments, the total weight of the composition comprises about 43 g. In some embodiments, the composition comprises citric acid.

Described herein is a method for improving the mouthfeel and minimizing the oral drying effect of a laxative, comprising the steps of: mixing the laxative with one or more USP/NF grade food ingredients wherein at least one of the one or more USP/NF grade food ingredients is a solid form at room temperature and an essentially liquid form at body temperature. In some embodiments, the laxative comprises at least 10 g of PEG 3350. In some embodiments, the laxative comprises at least 12 g of PEG 3350. In some embodiments, the laxative comprises PEG 3350. In some embodiments, the laxative comprises at least 15 g of PEG 3350. In some embodiments, the laxative comprises at least 20 g of PEG 3350. In some embodiments, the laxative comprises at least 25 g of PEG 3350. In some embodiments, the laxative comprises at least 30 g of PEG 3350. In some embodiments, the laxative comprises at least 35 g of PEG 3350. In some embodiments, the laxative comprises at least 40 g of PEG 3350. In some embodiments, the laxative comprises at least 45 g of PEG 3350. In some embodiments, the composition comprises about 50% PEG 3350 by weight. In some embodiments, the composition comprises about 55% PEG 3350 by weight. In some embodiments, the composition comprises about 60% PEG 3350 by weight. In some embodiments, the composition comprises about 65% PEG 3350 by weight. In some embodiments, the composition comprises about 70% PEG 3350 by weight. In some embodiments, the composition comprises about 75% PEG 3350 by weight. In some embodiments, the composition comprises about 80% PEG 3350 by weight. In some embodiments, the total weight of the composition comprises about 65 g. In some embodiments, the total weight of the composition comprises about 43 g. In some embodiments, the method comprises mixing the laxative and the one or more USP/NF food ingredients with citric acid.

### DETAILED DESCRIPTION

Described herein are compositions for purging the GI tract of a subject together with methods of manufacture of said compositions.

### Definitions

The term “subject” as used herein may refer to a human subject or any animal subject.

The term “USP/NF grade” as used herein refers to the standards as listed in monographs found in either the United States Pharmacopeia (USP) or the National Formulary (NF).

Compositions Comprising USP/NF Grade Ingredients

A USP/NF grade ingredient or chemical comprises an ingredient or chemical that meets the standards as listed in monographs found in the United States Pharmacopeia or the National Formulary. A monograph includes the name of the ingredient or preparation, the definition, packaging, storage, and labeling requirements, and the specification. The specification consists of a series of tests, procedures for the tests, and acceptance criteria. In some embodiments, a USP/NF grade food ingredient comprises granulated sucrose.

Described herein are compositions that comprise improved modes of delivery of laxative to a subject in order to, for example, prepare the bowel of the subject for a medical or surgical procedure or relieve constipation in the subject. In some embodiments, a composition as described herein comprises a laxative together with one or more USP/NF grade ingredients. In some embodiments, a USP/NF grade food ingredient comprises a sweetener. In some embodiments a USP/NF grade sweetener comprises a syrup. In some embodiments, a USP/NF grade sweetener comprises invert syrup. In some embodiments, a USP/NF grade sweetener comprises erythritol. In some embodiments, a USP/NF grade sweetener comprises maltodextrin. In some embodiments, a USP/NF grade sweetener comprises dextrin. In some embodiments, a USP/NF grade ingredient comprises a texturizer. In some embodiments, a USP/NF grade food ingredient comprises cocoa butter. In some embodiments, a USP/NF grade food ingredient comprises malic acid. In some embodiments, a USP/NF grade food ingredient comprises citric acid. In some embodiments, a USP/NF grade food ingredient comprises lemon oil. In some embodiments, a USP/NF grade food ingredient comprises vanilla flavor. In some embodiments, the laxative comprises a USP/NF grade laxative. Compositions described herein containing USP/NF grade foods and laxatives are beneficial in that, for example, such compositions provide for improved mouthfeel. The use of USP/NF food ingredients in the compositions described herein is also beneficial in that, for example, it provides the ability to include higher amounts of laxative in said compositions than would be possible if non-USP/NF ingredients were used.

Formulations

In some embodiments of the compositions described herein, a laxative and one or more food ingredients form an essentially homogeneous mixture. In some embodiments, the composition comprises at least 5 g of PEG 3350. In some embodiments, the laxative comprises PEG 3350. In some embodiments, the composition comprises at least 10 g of PEG 3350. In some embodiments, the composition comprises at least 12 g of PEG 3350. In some embodiments, the composition comprises at least 15 g of PEG 3350. In some embodiments, the composition comprises at least 20 g of PEG 3350. In some embodiments, the composition comprises at least 25 g of PEG 3350. In some embodiments, the composition comprises at least 30 g of PEG 3350. In some embodiments, the composition comprises at least 35 g of PEG 3350. In some embodiments, the composition comprises at least 40 g of PEG 3350. In some embodiments, the composition comprises at least 45 g of PEG 3350. In some embodiments, the composition comprises at least 50 g of PEG 3350. In some embodiments, the composition comprises at least 55 g of PEG 3350. In some embodiments, the composition comprises at least 60 g of PEG 3350. In some embodiments, the composition comprises at least 65 g of PEG 3350. In some embodiments, the composition comprises at least 70 g of PEG 3350. In some embodiments, the composition comprises at least 75 g of PEG 3350. In some embodiments, the composition comprises at least 80 g of PEG 3350. In some embodiments, the composition comprises at least 85 g of PEG 3350. In some embodiments, the composition comprises at least 90 g of PEG 3350. In some embodiments, the composition comprises at least 95 g of PEG 3350. In some embodiments, the composition comprises at least 100 g of PEG 3350. In some embodiments, the composition comprises at least 105 g of PEG 3350. In some embodiments, the composition comprises at least 110 g of PEG 3350. In some embodiments, the composition comprises at least 115 g of PEG 3350. In some embodiments, the composition comprises at least 120 g of PEG 3350. In some embodiments, the composition comprises at least 125 g of PEG 3350. In some embodiments, the composition comprises at least 130 g of PEG 3350. In some embodiments, the composition comprises at least 130 g of PEG 3350. In some embodiments, the composition comprises at least 135 g of PEG 3350. In some embodiments, the composition comprises at least 140 g of PEG 3350. In some embodiments, the composition comprises at least 145 g of PEG 3350. In some embodiments, the composition comprises at least 150 g of PEG 3350. In some embodiments, the composition comprises at least 155 g of PEG 3350. In some embodiments, the composition comprises at least 160 g of PEG 3350. In some embodiments, the composition comprises at least 165 g of PEG 3350. In some embodiments, the composition comprises at least 170 g of PEG 3350. In some embodiments, the composition comprises at least 175 g of PEG 3350. In some embodiments, the composition comprises at least 180 g of PEG 3350. In some embodiments, the composition comprises at least 185 g of PEG 3350. In some embodiments, the composition comprises at least 190 g of PEG 3350. In some embodiments, the composition comprises at least 195 g of PEG 3350. In some embodiments, the composition comprises at least 200 g of PEG 3350.

In some embodiments, the composition comprises about 50% PEG 3350 by weight. In some embodiments, the composition comprises about 55% PEG 3350 by weight. In some embodiments, the composition comprises about 60% PEG 3350 by weight. In some embodiments, the composition comprises about 65% PEG 3350 by weight. In some embodiments, the composition comprises about 70% PEG 3350 by weight. In some embodiments, the composition comprises about 75% PEG 3350 by weight. In some embodiments, the composition comprises about 80% PEG 3350 by weight. In some embodiments, the total weight of the composition comprises about 20 g. In some embodiments, the total weight of the composition comprises about 25 g. In some embodiments, the total weight of the composition comprises about 30 g. In some embodiments, the total weight of the composition comprises about 35 g. In some embodiments, the total weight of the composition comprises about 40 g. In some embodiments, the total weight of the composition comprises about 45 g. In some embodiments, the total weight of the composition comprises about 50 g. In some embodiments, the total weight of the composition comprises about 55 g. In some embodiments, the total weight of the composition comprises about 60 g. In some embodiments, the total weight of the composition comprises about 65 g. In some embodiments, the total weight of the composition comprises about 70 g.

In some embodiments, a composition as described herein comprises a solid food containing a laxative. In some embodiments, a composition as described herein comprises a liquid containing a laxative. Liquids as described herein, in some embodiments, comprise powders that are dissolved in said liquids. In some embodiments, a composition as described herein comprises a bar. In some embodiments, a composition as described herein comprises a shake or a fruit-flavored beverage. In some embodiments, a composition as described herein comprises a smoothie.

Modular Dosing

In some embodiments, one or more of the compositions described herein are provided in a modular fashion, for example, as part of a kit. When more than one composition as described herein is provided in a kit, a greater amount of laxative is delivered to a subject such as for the purpose of purging the bowel of the subject. For example, a kit in some embodiments contains a shake comprising of 300 g of laxative and a bar comprising 30 g of laxative to be eaten by a subject at one time thus providing a total of 330 g of laxative to the subject in one single dose.

Modular dose delivery also allows for a total targeted goal of laxative to be delivered over a longer period of time. For example, in some embodiments, two or more of the compositions described herein are provided to a subject in a kit and the subject ingests a first composition comprising 10 g of PEG 3350 in the morning with breakfast, and then the subject subsequently ingests a second composition comprises 15 g of PEG 3350 an hour or two later. In this way, a subject consumes a total amount of PEG 3350 equal to 25 g over a longer period of time as compared to ingesting the entire 25 g at once. This modular dosing is beneficial in that, for example, the effect of providing the PEG 3350 over time on the colon is more continuous and sustained. In some embodiments, modular dosing using the compositions described herein occurs in the course of one day. In some embodiments, modular dosing using the compositions described herein occurs in the course of 3 days. In some embodiments, modular dosing using the compositions described herein occurs in the course of one week. In some embodiments, modular dosing using the compositions described herein occurs in the course of 2 weeks. In some embodiments, modular dosing using the compositions described herein occurs in the course of one month. In some embodiments, modular dosing using the compositions described herein occurs in the course of more than one month.

Modular dosing is achieved with any of the compositions types described herein either individually or in combination with other types. For example, in some embodiments two bars, each comprising a composition as described herein, are provided to a subject, and the subject ingests the first bar and subsequently ingests the second bar 3 hours later. For example, in some embodiments a liquid and a solid food, each comprising a composition as described herein, are provided to a subject, and the subject ingests the liquid and subsequently ingests the solid food 1 hour later.

Methods of Manufacture

Described herein is a method for manufacturing a laxative composition. In some embodiments, the method comprises heating a USP/NF grade fat to a temperature at which the USP/NF grade fat becomes essentially liquefied then mixing the essentially liquefied USP/NF grade fat with a syrup and a laxative. In some embodiments, the temperature at which the USP/NF grade fat becomes essentially liquefied comprises about 95 degrees Fahrenheit. In some embodiments, the temperature at which the USP/NF grade fat becomes essentially liquefied comprises about 90 degrees Fahrenheit. In some embodiments, the temperature at which the USP/NF grade fat becomes essentially liquefied comprises about 85 degrees Fahrenheit. In some embodiments, the temperature at which the USP/NF grade fat becomes essentially liquefied comprises about 80 degrees Fahrenheit. In some embodiments, the temperature at which the USP/NF grade fat becomes essentially liquefied comprises about 75 degrees Fahrenheit. In some embodiments, the syrup comprises an invert syrup. In some embodiments, the laxative comprises PEG 3350. In some embodiments, the PEG 3350 comprises a powder having a coarse grain texture. In some embodiments, the liquefied USP/NF grade fat is allowed to cool to a temperature of about 80 degrees before the syrup and the laxative are mixed with it. In some embodiments, the liquefied USP/NF grade fat is allowed to cool to a temperature of about 75 degrees before the syrup and the laxative are mixed with it. In some embodiments, the liquefied USP/NF grade fat is allowed to cool to a temperature of about 70 degrees before the syrup and the laxative are mixed with it. In some embodiments, the liquefied USP/NF grade fat is allowed to cool to a temperature of about 65 degrees before the syrup and the laxative are mixed with it. In some embodiments, the liquefied USP/NF grade fat is allowed to cool to a temperature of about 60 degrees before the syrup and the laxative are mixed with it. In some embodiments, the syrup is mixed with the essentially liquefied USP/NF grade fat before it cools. In some embodiments, the syrup is mixed with the essentially liquefied USP/NF grade fat before the laxative is mixed in. In some embodiments, the essentially liquefied USP/NF grade fat, the syrup, and the laxative are mixed together to form an essentially homogeneous mixture. In some embodiments, the mixture is allowed to cool and formed into a solid that may be further divided into individual bars. In some embodiments, a USP/NF grade fat comprises cocoa butter. In some embodiments, a USP/NF grade fat comprises a USP/NF grade fat that is contained in a food. In some embodiments, a USP/NF grade fat comprises a USP/NF grade ingredient. In some embodiments, a syrup comprises a USP/NF grade ingredient. In some embodiments, a laxative comprises a USP/NF grade laxative.

The methods of manufacture described herein are beneficial, for example, because they prevent a laxative incorporated into the compositions described herein from being heated to high temperatures, while still resulting in homogeneous mixtures of laxatives with other ingredients and composition elements. Heating of laxatives such as, for example, PEG 3350 to high temperatures can lead to PEG break down and the formation of PEG degradation products such as, for example, Diethylene Glycol and Ethylene Glycol, which can be dangerous if ingested. Because the laxatives are not excessively heated in the manufacturing process the generation of, potentially harmful, laxative degradation products is prevented.

Improved Texture and Mouthfeel

Described herein is a composition containing a laxative, wherein the composition is configured to provide an improved mouth feel and minimize oral drying associated with, for example, osmotic laxatives. In some embodiments, a composition comprises an essentially homogeneous mixture of a laxative and a USP/NF grade fat, wherein the USP/NF grade fat is an essentially solid form at room temperature and an essentially liquid form at body temperature. In some embodiments, a composition comprises sufficient USP/NF grade fat to coat the composition as well as the palate of a subject when it is ingested by the subject thus blocking the effects of a laxative on the mucosa of the mouth and pharynx of the subject. In some embodiments, the USP/NF grade fat coats the composition by becoming an essentially liquid form when in the mouth and pharynx of the subject, which are at body temperature. In some embodiments, a composition further comprises a food acid. In some embodiments, a food acid comprises citric acid. In some embodiments, a USP/NF grade fat that comprises an essentially solid form at room temperature and an essentially liquid form at body temperature comprises a USP/NF grade food. In some embodiments, a laxative comprises a USP/NF grade laxative. In some embodiments, a food acid comprises a USP/NF grade food acid.

In some embodiments a USP/NF grade fat as described herein comprises a USP/NF grade fat that holds together as a single mass at room temperature. In some embodiments, a USP/NF grade fat as described herein may be molded into an appropriate form without the use of heat or additional binders or emulsifiers.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

Table 1 shows the ingredients, weights, and percent by weight of an embodiment of a composition comprising one or more USP/NF food ingredients as described herein comprising a vanilla milkshake.

TABLE 1
Vanilla Milkshake-USP/NF

| | | Amount per |
|---|---|---|
| Ingredient | % | Unit (grams) |
| Polyethylene Glycol 3350 | 86.2% | 115.0 |
| Sucrose, granulated USP | 12.3% | 16.3 |
| Milky Vanilla Flavor Powder, | 1.5% | 2.0 |
| N&A #2713, David Michael | | |
| | 100.0% | 133.3 |

Table 2 shows the ingredients, weights, and percent by weight of an embodiment of a composition comprising one or more USP/NF food ingredients described herein comprising a strawberry-banana smoothie.

TABLE 2
Strawberry-Banana Smoothie-USP/NF

| | | Amount |
|---|---|---|
| | % By | per Unit |
| Ingredient | Weight | (grams) |
| Polyethylene Glycol 3350 | 93.9% | 116.6 |
| Sucrose, granulated USP | 5.6% | 7.0 |
| Strawberry Banana, N&A #3620, David Michael | 0.4% | 0.5 |
| Malic acid, USP | 0.1% | 0.1 |
| | 100.0% | 124.2 |

Table 3 shows the ingredients, weights, and percent by weight of an embodiment of a composition comprising one or more USP/NF food ingredients described herein comprising a lemon cooler bar.

TABLE 3
Lemon Cooler Bar-USF/NF #4 (43.1 g bar)

| | | Amount per |
|---|---|---|
| Ingredient | % By Weight | Unit (grams) |
| Polyethylene Glycol 3350 | 69.5% | 30.0 |
| Invert Syrup | 17.0% | 7.3 |
| Cocoa Butter | 12.6% | 5.4 |
| Milky Vanilla Flavor Powder, | 0.7% | 0.3 |
| N&A #2713, David Michael | | |
| Citric Acid | 0.1% | 0.1 |
| Lemon Oil | 0.1% | 0.0 |
| | 100.0% | 43.1 |

Table shows the ingredients, weights, and percent by weight of an embodiment of a composition comprising one or more USP/NF food ingredients described herein comprising an alternative lemon cooler bar.

TABLE 4
Lemon Bar USP/NF

| | | Amount |
|---|---|---|
| | % By | per Unit |
| Ingredient | weight | (grams) 24.4 |
| Polyethylene Glycol 3350 | 69.8% | 17.023 |
| Invert Syrup | 17.0% | 4.142 |
| Cocoa Butter | 12.4% | 3.018 |
| Flavor, Milky Vanilla N&A | 0.7% | 0.171 |
| #2713, David Michael | | |
| Citric Acid | 0.1% | 0.029 |
| Lemon Oil | 0.1% | 0.016 |
| | 100.0% | 24.400 |

Table 5 shows an embodiment of a bar making process.

TABLE 5
bar making process:
melt cocoa butter (around 92° F.)
blend invert syrup and liquid flavors with melted cocoa butter
pre-blend dry ingredients-citric acid, vanilla flavor powder with PEG 3350
mix both portions together
form into bars and cool

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition containing PEG 3350 and one or more USP/NF grade food ingredients, wherein the composition comprises an essentially homogeneous mixture of the PEG 3350 and the one or more USP/NF grade food ingredients.

2. The composition of claim 1, wherein the composition comprises at least 10 g of PEG 3350.

3. The composition of claim 1, wherein the composition comprises at least 12 g of PEG 3350.

4. The composition of claim 1, wherein the composition comprises at least 15 g of PEG 3350.

5. The composition of claim 1, wherein the composition comprises at least 20 g of PEG 3350.

6. The composition of claim 1, wherein the composition comprises at least 25 g of PEG 3350.

7. The composition of claim 1, wherein the composition comprises at least 30 g of PEG 3350.

8. The composition of claim 1, wherein the composition comprises at least 35 g of PEG 3350.

9. The composition of claim 1, wherein the composition comprises at least 40 g of PEG 3350.

10. The composition of claim 1, wherein the composition comprises at least 45 g of PEG 3350.

11. The composition of claim 1, wherein the composition comprises about 50% PEG 3350 by weight.

12. The composition of claim 1, wherein the composition comprises about 55% PEG 3350 by weight.

13. The composition of claim 1, wherein the composition comprises about 60% PEG 3350 by weight.

14. The composition of claim 1, wherein the composition comprises about 65% PEG 3350 by weight.

15. The composition of claim 1, wherein the composition comprises about 70% PEG 3350 by weight.

16. The composition of claim 1, wherein the composition comprises about 75% PEG 3350 by weight.

17. The composition of claim 1, wherein the composition comprises about 80% PEG 3350 by weight.

18. The composition of claim 1, wherein the total weight of the composition comprises about 65 g.

19. The composition of claim 1, wherein the total weight of the composition comprises about 43 g.

20. A method for manufacturing a composition comprising the steps of:
heating one or more USP/NF grade food ingredients to a temperature of about 90 degrees Fahrenheit until the one or more USP/NF grade food ingredients is essentially liquefied; and
mixing the essentially liquefied one or more USP/NF grade food ingredients with a laxative.

21. The method of claim 20, wherein at least one of the one or more USP/NF grade food ingredients comprises a pure fat.

22. The method of claim 20, wherein at least one of the one or more USP/NF grade food ingredients is a solid at room temperature.

23. The method of claim 22, wherein at least one of the one or more USP/NF grade food ingredients comprises cocoa butter.

24. The method of claim 20, wherein the composition comprises syrup.

25. The method of claim 20, wherein the step of mixing comprises mixing the one or more USP/NF grade food ingredients with the laxative at a temperature below 70 degrees Fahrenheit.

26. The method of claim 20, wherein the laxative comprises PEG 3350.

27. The method of claim 26, wherein the amount of PEG 3350 is at least 10 g.

28. The method of claim 26, wherein the amount of PEG 3350 is at least 12 g.

29. The method of claim 26, wherein the amount of PEG 3350 is at least 15 g.

30. The method of claim 26, wherein the amount of PEG 3350 is at least 20 g.

31. The method of claim 26, wherein the amount of PEG 3350 is at least 25 g.

32. The method of claim 26, wherein the amount of PEG 3350 is at least 30 g.

33. The method of claim 26, wherein the amount of PEG 3350 is at least 35 g.

34. The method of claim 26, wherein the amount of PEG 3350 is at least 40 g.

35. The method of claim 26, wherein the amount of PEG 3350 is at least 45 g.

36. The method of claim 26, wherein the composition comprises about 50% PEG 3350 by weight.

37. The method of claim 26, wherein the composition comprises about 55% PEG 3350 by weight.

38. The method of claim 26, wherein the composition comprises about 60% PEG 3350 by weight.

39. The method of claim 26, wherein the composition comprises about 65% PEG 3350 by weight.

40. The method of claim 26, wherein the composition comprises about 70% PEG 3350 by weight.

41. The method of claim 26, wherein the composition comprises about 75% PEG 3350 by weight.

42. The method of claim 26, wherein the composition comprises about 80% PEG 3350 by weight.

43. The method of claim 26, wherein the total weight of the composition comprises about 65 g.

44. The method of claim 26, wherein the total weight of the composition comprises about 43 g.

45. A composition comprising an essentially homogeneous mixture of a laxative and one or more USP/NF food ingredients wherein the at least one of the one or more USP/NF food ingredients are in essentially solid form at room temperature and essentially liquid form at body temperature.

46. The composition of claim 45, wherein the laxative comprises PEG 3350.

47. The composition of claim 46, wherein the amount of PEG 3350 is at least 10 g.

48. The composition of claim 46, wherein the amount of PEG 3350 is at least 12 g.

49. The composition of claim 46, wherein the amount of PEG 3350 is at least 15 g.

50. The composition of claim 46, wherein the amount of PEG 3350 is at least 20 g.

51. The composition of claim 46, wherein the amount of PEG 3350 is at least 25 g.

52. The composition of claim 46, wherein the amount of PEG 3350 is at least 30 g.

53. The composition of claim 46, wherein the amount of PEG 3350 is at least 35 g.

54. The composition of claim 46, wherein the amount of PEG 3350 is at least 40 g.

55. The composition of claim 46, wherein the amount of PEG 3350 is at least 45 g.

56. The composition of claim 46, wherein the composition comprises about 50% PEG 3350 by weight.

57. The composition of claim 46, wherein the composition comprises about 55% PEG 3350 by weight.

58. The composition of claim 46, wherein the composition comprises about 60% PEG 3350 by weight.

59. The composition of claim 46, wherein the composition comprises about 65% PEG 3350 by weight.

60. The composition of claim 46, wherein the composition comprises about 70% PEG 3350 by weight

61. The composition of claim 46, wherein the composition comprises about 75% PEG 3350 by weight.

62. The composition of claim 46, wherein the composition comprises about 80% PEG 3350 by weight.

63. The composition of claim 46, wherein the total weight of the composition comprises about 65 g.

64. The composition of claim 46, wherein the total weight of the composition comprises about 43 g.

65. The composition of claim 46, further comprising citric acid.

66. A method for improving the mouthfeel and minimizing the oral drying effect of a laxative, comprising the steps of:
mixing the laxative with one or more USP/NF grade food ingredients wherein at least one of the one or more USP/NF grade food ingredients is a solid form at room temperature and an essentially liquid form at body temperature.

67. The method of claim 66, wherein the laxative comprises PEG 3350.

68. The method of claim 67, wherein the amount of PEG 3350 is at least 10 g.

69. The method of claim 67, wherein the amount of PEG 3350 is at least 12 g.

70. The method of claim 67, wherein the amount of PEG 3350 is at least 15 g.

71. The method of claim 67, wherein the amount of PEG 3350 is at least 20 g.

72. The method of claim 67, wherein the amount of PEG 3350 is at least 25 g.

73. The method of claim 67, wherein the amount of PEG 3350 is at least 30 g.

74. The method of claim 67, wherein the amount of PEG 3350 is at least 35 g.

75. The method of claim 67, wherein the amount of PEG 3350 is at least 40 g.

76. The method of claim 67, wherein the amount of PEG 3350 is at least 45 g.

77. The method of claim 67, wherein the composition comprises about 50% PEG 3350 by weight.

78. The method of claim 67, wherein the composition comprises about 55% PEG 3350 by weight.

79. The method of claim 67, wherein the composition comprises about 60% PEG 3350 by weight.

80. The method of claim 67, wherein the composition comprises about 65% PEG 3350 by weight.

81. The method of claim 67, wherein the composition comprises about 70% PEG 3350 by weight.

82. The method of claim 67, wherein the composition comprises about 75% PEG 3350 by weight.

83. The method of claim 67, wherein the composition comprises about 80% PEG 3350 by weight.

84. The method of claim 67, wherein the total weight of the composition comprises about 65 g.

85. The method of claim 67, wherein the total weight of the composition comprises about 43 g.

86. The method of claim 67, further comprising mixing the laxative and the one or more USP/NF grade food ingredients with citric acid.
